(19) United States
(12) Patent Application Publication
(10) Pub. No.: US 2003/0120144 A1
(43) Pub. Date: Jun. 26, 2003

(54) Intrapericardial temperature measurement device and method

(76) Inventors: James R. Grabek, Minneapolis, MN (US); Michael Hoey, Shoreview, MN (US)

Correspondence Address: Beck & Tysver, P.L.L.C., Suite 100, 2900 Thomas Avenue S., Minneapolis, MN, 55416, US

(21) Appl. No.: 10/295,413

(22) Filed: Nov. 15, 2002

Related U.S. Application Data
(60) Provisional application No. 60/332,356, filed on Nov. 16, 2001.

(51) Int. Cl. A61B 5/05

(52) U.S. Cl. 600/407

(57) ABSTRACT

The method of the invention uses a thermal imager within the pericardial space to monitor the progress of an ablation procedure occurring within the heart chamber.

### CROSS REFERENCE TO RELATED APPLICATIONS

This application claims the benefit of and incorporates by reference U.S. Provisional Application 60/332,356 entitled Intrapericardial Microbolometer filed Nov. 16, 2001.

### FIELD OF THE INVENTION

The present invention relates to temperature measurement in living tissue. And more particularly to a system for monitoring and guiding a cardiac ablation procedure.

### BACKGROUND OF THE INVENTION

Temperature measurement of tissue and organs in vivo is widely practiced both for diagnostic and therapeutic uses. A variety of temperature measurement technologies have been applied to living systems ranging from simple contact thermometers to MRI based temperature-measuring system.

The use of radio frequency current to injure cardiac tissue is a well-known therapy to interrupt cardiac arrhythmias.

### SUMMARY OF THE INVENTION

The present invention relates to the use of thermometry and remote temperature measurement devices to monitor a surgical procedure. In contrast to conventional cardiac ablation techniques the present invention proposes the use of contact or non-contact thermometry to measure the size and location of lesions in cardiac tissue created by ablation from a location outside the heart in the pericardial space.

### BRIEF DESCRIPTION OF DRAWINGS

In the drawing identical reference numerals indicate identical structure. wherein:

FIG. 1 shows non-contact thermometry in a heart with an intact pericardium.

FIG. 2 shows a contact temperature monitoring system.

### DETAILED DESCRIPTION

As presently practiced cardiac ablation occurs when radio frequency energy is delivered to a catheter 10 in the blood pool within a cardiac chamber such as the atrium 12. In operation the physician places the electrode 10 in contact with the cardiac tissue and a radio frequency current from a generator 24 is delivered between poles on the catheter resulting in tissue damage adjacent the catheter.

As a therapy the injured tissue interrupts electrical conduction through the heart tissue interrupting electrical arthymias. Successful ablation to interrupt arthymia requires that the lesion size be both controlled and known to the practitioner.

A lesion which is too small or too short cannot successfully intercept electrical conduction permitting the arthymia to continue.

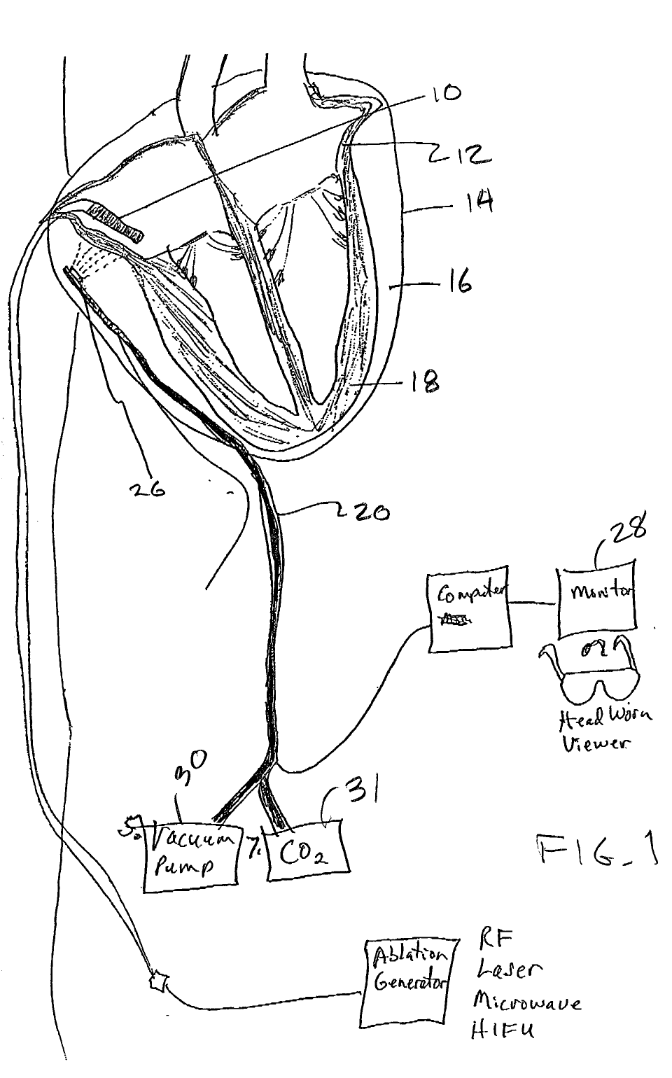

FIG. 1 shows an ablation electrode in a blood pool within the atrium of the heart laying a lesion along the wall of the heart. Within the pericardial space 16 a microbolometer or other non-contact thermal imaging sensors is positioned to monitor and measure temperature changes on the surface of the heart.

It is anticipated that a sharp contrast in temperature profile will be observed when the RF catheter is producing a lesion. Knowledge of the size and length of the lesion can be interpreted by the physician as part of the therapy. The use of a non-contact thermal imaging device allow the physician to monitor the progress of lesion formation with the computer monitor. Many thermal imaging techniques are workable but mocrobolometry is preferred. To improve performance of the microbolometer it is preferred to remove pericardial fluid 16 with a vacuum system introduced into the pericardial space 14. It is also preferred to inflate the pericardium with CO2 from dispenser 31, to increase the range of vision and to eliminate the quenching effect of the fluid.

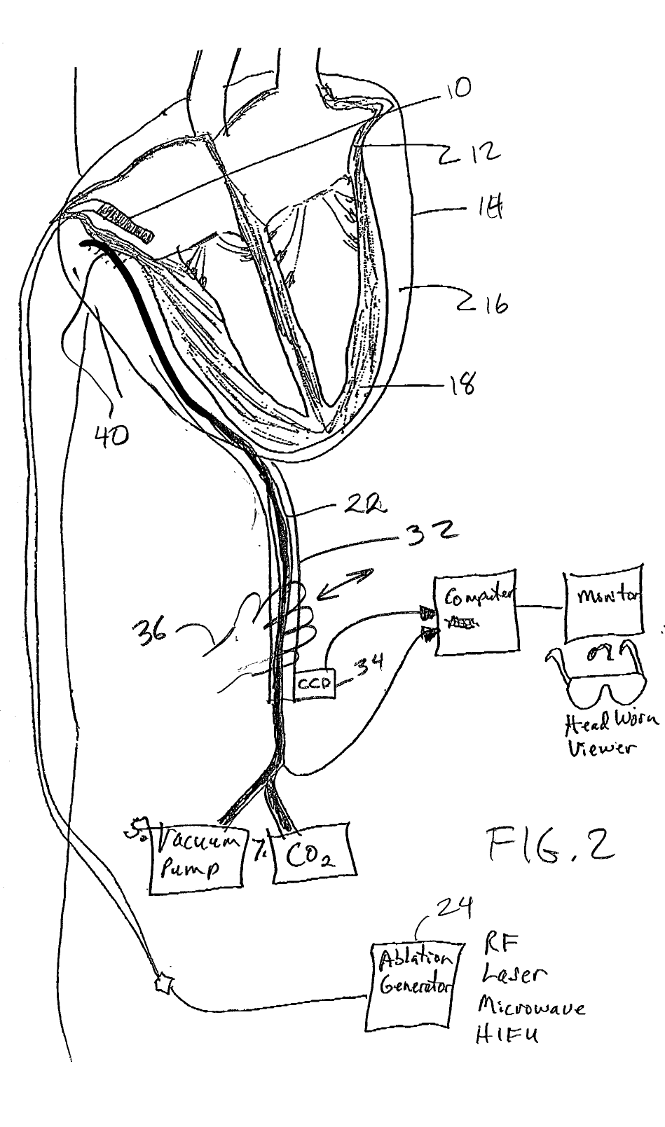

FIG. 2 shows an alternate in-contact system with a contact thermometry which may be thermister based or based on a movable miniature MRI antenna. The contact sensor 40is moved along the surface of the heart by manipulation are a physician indicated in the figure by hand 36. It is preferred but not required to visualize the procedure with a laparoscope 32 having a CCD camera 34 for displaying an image on the computer and monitor. Once again it is preferred to drain the pericardial fluid and replace it with a gas such as CO2.

As seen in the figure, it is desirable to have a laparoscope or other optical visualization device present in the pericardial space to help manipulate and position the microbolometer FIG. 2 shows a contact thermometry device which may be thermometry based or an ultrasound transducer. Thermister based thermometry simply measures the tissue contact at the site of the catheter while an ultrasound transducer notes the change in reflectance caused by the thermal damage to tissue. In the case of ultrasound the reflection is based both upon change in the tissue's characteristics as well as the temperature of the tissue. In operation the physician will move the RF catheter creating a lesion while the monitoring system will determine the size, location, shape and direction of the lesion and provide this information to the physician through a monitor or other physician interface.

What is claimed is:

1. A method of determining the size of a lesion comprising the steps of:
inserting a non-contact thermal imager into the pericardial space;
inserting an ablation catheter into the heart at a location near said imager;
activating said ablation catheter to make a lesion;
observing the thermal damage associated with ablation from said thermal imager;
determining the size of the lesion from said observation.
